(19) United States
(12) Patent Application Publication
(10) Pub. No.: US 2014/0052272 A1
(43) Pub. Date: Feb. 20, 2014

(54) COMFORT STENTS

(71) Applicant: Boston Scientific Scimed, Inc., Maple Grove, MN (US)

(72) Inventors: Raymond G. Amos, JR., Spencer, IN (US); Timothy P. Harrah, Cambridge, MA (US); Bryan Kostelac, Camby, IN (US); Kenneth P. Reever, Hopedale, MA (US)

(73) Assignee: Boston Scientific Scimed, Inc., Maple Grove, MN (US)

(21) Appl. No.: 13/968,002

(22) Filed: Aug. 15, 2013

Related U.S. Application Data
(60) Provisional application No. 61/691,010, filed on Aug. 20, 2012.

(51) Int. Cl. A61M 27/00

(52) U.S. Cl. CPC A61M 27/008
USPC 623/23.69; 623/23.7

(57) ABSTRACT

In one embodiment, a stent includes a retention portion and an elongate portion. The retention portion is configured to help retain the stent in place within a body of a patient. The elongate portion extends from the retention portion. The elongate portion has a sidewall defining a lumen and the outer surface of the sidewall includes a pattern. The sidewall has a first section and second section. The first section of the sidewall has a first thickness. The second section of the sidewall has a second thickens different than the first thickness.

### CROSS-REFERENCE TO RELATED APPLICATION

This application is a Nonprovisional of, and claims priority to, U.S. Patent Application No. 61/691,010, filed on Aug. 20, 2012, entitled “COMFORT STENTS”, which is incorporated by reference herein in its entirety.

### TECHNICAL FIELD

This disclosure relates generally to stents, including ureteral stents, that are configured to extend from one location within a body of a patient to another location within the body of the patient.

### BACKGROUND

Medical devices, such as stents, are often used to drain fluids within a patient's body. For example, ureteral stents can be used to assist the drainage of fluids through the urinary system of a patient. Some known ureteral stents include tubular members and are configured to assist the drainage of fluid from one part of the urinary system to another part of the urinary system. Some known ureteral stents are configured to extend from a patient's kidney to a patient's bladder. Such known ureteral stents assist to drain fluid from the patient's kidney to the patient's bladder.

Regions of the urinary system are particularly sensitive and are prone to irritation by foreign objects. Thus, to avoid patient irritation and pain, it may be advantageous to provide urinary stents that are soft of flexible such that they avoid or lightly contact the sensitive regions. The stents, however, should be stiff or rigid enough such that the stent may be placed within the body of the patient and provide the appropriate drainage functions. Thus, it would be advantageous to provide a stent that was sufficiently rigid to be inserted and provide the drainage functions, yet would be soft or flexible enough to potentially avoid some patient pain or discomfort.

Additionally, some known medical devices, such as stents, are stiff or rigid. Such medical devices may not flex or conform to a body of a patient or movements of the body of the patient. This may cause pain or discomfort to a patient. Thus, it would be advantageous to provide a medical device or stent that is configured to flex and bend to conform to a body of a patient or to bodily movements of the patient.

### SUMMARY

In one embodiment, a stent includes a retention portion and an elongate portion. The retention portion is configured to help retain the stent in place within a body of a patient. The elongate portion extends from the retention portion. The elongate portion has a sidewall defining a lumen. The sidewall has a first section and second section. The first section of the sidewall has a first thickness. The second section of the sidewall has a second thickens different than the first thickness.

In another embodiment, a stent includes a retention portion and an elongate portion. The retention portion is configured to help retain the stent in place within a body of a patient. The elongate portion extends from the retention portion. The elongate portion has a first member and a second member. The first member is devoid of a lumen. The second member is devoid of a lumen.

In another embodiment, a stent includes a retention portion and an elongate portion. The retention portion is configured to help retain the stent in place within a body of a patient. The elongate portion extends from the retention portion and has an inflated or expanded configuration and a deflated, contracted, or nominal configuration. The elongate portion has a sidewall defining a lumen extending from a first end portion of the elongate portion to a second end portion of the elongate portion. The sidewall defines a chamber. The chamber is configured to receive a fluid to place the elongate portion in its inflated or expanded configuration.

### BRIEF DESCRIPTION OF THE DRAWINGS

FIG. 1A is a schematic illustration of a medical device according to an embodiment of the invention.

FIG. 1B is a schematic illustration of the medical device of FIG. 1A disposed within a body of a patient.

FIG. 2 is a perspective view of a medical device according to an embodiment of the invention.

FIG. 3 is a cross-sectional view of the medical device of FIG. 2 taken along line A-A of FIG. 2.

FIG. 4 is a cross-sectional view of the medical device of FIG. 2 taken along line B-B of FIG. 2.

FIG. 5 is a perspective view of a medical device according to an embodiment of the invention.

FIG. 6 is a cross-sectional view of the medical device of FIG. 5 taken along line C-C of FIG. 5.

FIG. 7 is a perspective view of a medical device according to an embodiment of the invention.

FIG. 8 is a perspective view of a medical device according to an embodiment of the invention.

FIG. 9 is a cross-sectional view of the medical device of FIG. 8 taken along line D-D of FIG. 8.

FIGS. 10-13 are perspective views of medical devices according to embodiments of the invention.

FIG. 14 is a perspective view of a medical device in an expanded configuration according to an embodiment of the invention.

FIG. 15 is a perspective view of the medical device of FIG. 14 in a collapsed configuration.

FIG. 16 is a cross-sectional view of the medical device of FIG. 14 taken along line T-T of FIG. 14.

FIG. 17 is a perspective view of a medical device according to an embodiment of the invention.

### DETAILED DESCRIPTION

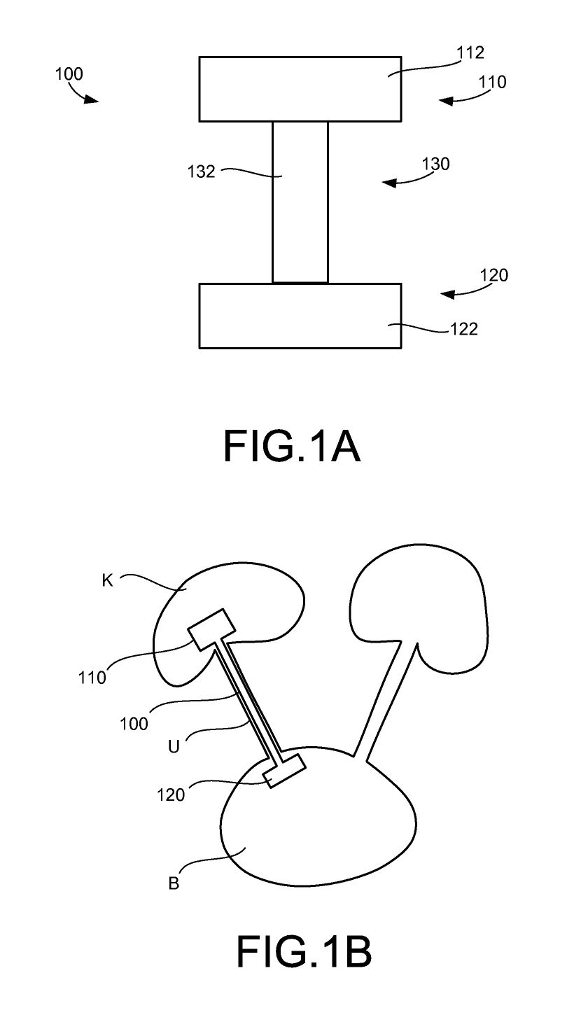

FIG. 1A is a schematic illustration of a medical device 100 according to an embodiment of the invention. FIG. 1B is a schematic illustration of the medical device 100 disposed within a body of a patient.

The medical device 100 is configured to be disposed within a body of a patient. For example, in some embodiments, the medical device 100 is a stent and is configured to be disposed within a body of a patient, such as a male patient, a female patient, or any other mammal or other type of patient. As illustrated in FIG. 1B, in some embodiments, the medical device 100 is configured to be disposed within a urinary tract of a patient. Specifically, the medical device 100 may be configured to be placed or disposed within the body of the patient such that the medical device 100 extends from a kidney K of the patent, through a ureter U, and into a bladder B of the patient. In other embodiments, the medical device 100 may be placed within the body of the patient such that it does not extend from the kidney K to the bladder B. For example, the medical device 100 may extend from the kidney K to a location within the ureter U of the patient. In other embodiments, the medical device 100 may extend from a location within the body of the patient to a location outside of the body of the patient. In yet further embodiments, the medical device 100 may be configured to be disposed in portions or locations within the body of the patient other than the urinary tract of the patient.

In some embodiments, the medical device 100 is a stent and is configured to help facilitate drainage of fluid from one location within the body of the patient to another location within the body of the patient. For example, in some embodiments, the stent includes or defines a lumen that is configured to pass or transport fluid, such as a bodily fluid, from one location within the body of the patient to another location within the body of the patient. In other embodiments, the stent is configured to be disposed within the body of the patient such that it helps retain or maintain a bodily lumen clear or free from blockages to help facilitate the passing of fluid from one location within the body of the patient to another location within the body of the patient. In some embodiments, the medical device 100 is configured to be placed or disposed within a urinary tract of a patient. In other embodiments, the medical device 100 is configured to be placed or disposed within other bodily lumens or ducts, including, but not limited to, a biliary duct or a pancreatic duct.

In one embodiment, the medical device 100 is a ureteral stent and includes a distal end portion 110, a proximal end portion 120, and a medial portion 130 disposed between the distal end portion 110 and the proximal end portion 120. In such an embodiment, as illustrated in FIG. 1B, the distal end portion 110 is configured to be disposed within a kidney K of a patient and the proximal end portion 120 is configured to be disposed within a bladder B of the patient. The medial portion 130 is configured to extend within a ureter U of the patient. In such an embodiment, the medical device 100 is configured to help facilitate drainage of fluids such as urine within the urinary tract of the patient. Specifically, in such an embodiment, the medical device 100 is configured to facilitate drainage from the kidney K of the patient to the bladder B of the patient.

In the illustrated embodiment, the medial portion 130 of the device 100 includes an elongate portion or member 132. In some embodiments, the elongate portion or member 132 of the medical device 100 defines a lumen. The lumen is configured to help facilitate the drainage of fluid within the body of the patient. Additionally, in some embodiments, side ports or openings are located along the length of the elongate portion or member 132. The side ports or openings communicate with the lumen and further facilitate the drainage of fluid within the body. In other embodiments, the elongate portion or member 132 is formed of a material that is configured to move or wick the fluid from one location within the body to another location within the body. In yet other embodiments, the elongate portion or member 132 is configured to be disposed within a bodily lumen (such as the ureter) and is configured to help retain the patency of the bodily lumen. In other words, in such embodiments, the elongate portion or member is configured to help keep the bodily lumen in an open state.

In the illustrated embodiment, the distal end portion 110 of the device 100 includes a retention portion or member 112. In some embodiments, the retention portion or member 112 extends from the elongated portion or member 132 and is configured to help retain the device 100 in place within the body of the patient. For example, in some embodiments, the retention portion or member 112 is configured to be placed within a kidney of the patient to help retain the medical device 100 in place within the urinary tract of the patient. In other embodiments, the medical device 100 is configured to be placed or disposed within a biliary duct or pancreatic duct of the patient. The retention portion or member 112 may have any shape or configuration. For example, in some embodiments, the retention portion or member 112 has a loop, coil, spiral, or helical shape. In other embodiments, the retention portion 112 or member has another shape. In some embodiments, the retention portion or member 112 may assume a collapsed or smaller configuration to facilitate the insertion of the device 100 into the body of the patient and may assume an expanded or larger configuration once the device 100 is correctly placed within the body of the patient.

In the illustrated embodiment, the proximal end portion 120 of the device 100 includes a retention portion or member 122. In some embodiments, the retention portion or member 122 extends from the elongated portion or member 132 and is configured to help retain the device 100 in place within the body of the patient. For example, in some embodiments, the retention portion or member 122 is configured to be placed within a bladder of the patient to help retain the medical device 100 in place within the urinary tract of the patient. The retention portion or member 122 may have any shape or configuration. For example, in some embodiments, the retention portion or member 122 has a loop, coil, spiral, or helical shape. In other embodiments, the retention portion 122 or member has another shape. In some embodiments, the retention portion or member 122 may assume a collapsed or smaller configuration to facilitate the insertion of the device 100 into the body of the patient and may assume an expanded or larger configuration once the device 100 is correctly placed within the body of the patient.

In some embodiments, the medical device 100 is configured to avoid contact with sensitive regions within the body of the patient. For example, in one embodiment, the medical device 100 is configured to avoid contact with the trigone region of the patient when the proximal end portion 120 of the medical device 100 is disposed within the bladder of a patient. In other embodiments, the medical device 100 is configured to shape or flex to the specific contours of the body of the patient or to the bodily moves of the patient. For example, in some embodiments, the elongate portion or member 132 is configured to flex or conform to the curves or shape of the ureter of the patient. In some embodiments, the device 100 has a rigidity or stiffness during the insertion of the device and a second rigidity or stiffness which is less rigid or stiff once the device 100 is placed within the body of the patient. For example, the device 100 may be formed of a material that is configured to or formulated to soften once the device 100 is placed within the body of the patient (such as a material that absorbs bodily fluid and softens once inserted into the body of the patient).

The medical device 100 may be placed within the body of a patient using any known placement method. For example, the medical device 100 may be placed within the urinary tract of a patient using a guidewire to guide the medical device 100 into place within the body of the patient. Additionally or alternatively, an insertion tube or delivery catheter may be used to place the medical device within the body of the patient. In some embodiments, the medical device 100 is delivered into the body of the patient percutaneously.

In some embodiments, the medical device 100 is constructed of a biocompatible material. For example, in some embodiments, the medical device 100 is constructed of a biocompatible plastic, such as, but not limited to, polyester, nylon based biocompatible polymers, polytetrafluoroethylene polymers, silicone polymers, polyurethane polymers, polyethylene polymers, and thermoplastic polymers. In one embodiment, the medical device is constructed of ethylene vinyl acetate.

The medical device 100 may be formed using any known construction method. For example, in some embodiments, the medical device 100, including the distal end portion 110, the medial portion 130, and the proximal end portion 120 are monolithic or unitarily formed. In such embodiments, the medical device 100 may be formed using, for example, an extrusion technique or an injection molding technique. In other embodiments, the medical device 100 is not monolithic or unitarily formed. In such embodiments, the different portions of the medical device 100 maybe coupled together using any known method, such as a melting or gluing method.

The medical device 100 may be sized to appropriately fit within the body of the patient. For example, in some embodiments, the medical device 100 is sized such that the distal end portion 110 may be disposed in a kidney of a patient, the proximal end portion 120 may be disposed in a bladder of a patient, and the medial portion 130 may be disposed within a ureter of a patient. The medical device 100 can be of any diameter. For example, in some embodiments, the medial portion 130 has an outer diameter of between 4 French and 12 French. In other embodiments, the outer diameter of the medial portion is smaller than 4 French or greater than 12 French.

In some embodiments, a portion of the medical device 100 may extend from the body of the patient once it is placed within the body of the patient. In such embodiments, the medical device 100 may be configured to help convey fluid from one portion of the body to a location outside of the body of the patient.

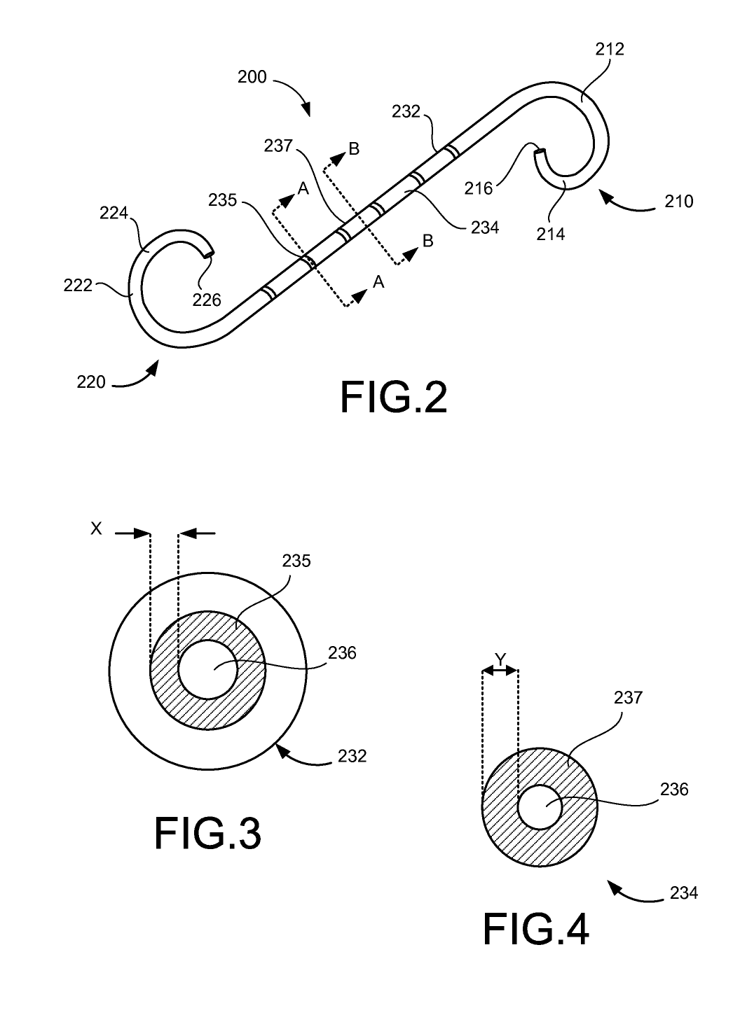

FIG. 2 is a perspective view of a medical device 200 according to an embodiment of the invention. FIG. 3 is a cross-section view of the medical device 200 taken along line A-A of FIG. 2. FIG. 4 is a cross-sectional view of the medical device 200 taken along line B-B of FIG. 2. The medical device 200 is a ureteral stent and is configured to be disposed within a urinary tract of a patient and help transport bodily fluids from a kidney of a patient to a bladder of the patient. In other embodiments, the medical device 200 is configured to be placed or disposed in another portion of the body, such as a biliary duct or a pancreatic duct. The medical device 200 includes a distal end portion 210, a proximal end portion 220, and a medial portion 230. In some embodiments, the medical device 200 may be placed within a body of a patient such that the distal end portion 210 may be disposed within the kidney of the patient, the proximal end portion 220 may be disposed within the bladder of the patient, and the medial portion 230 may be disposed within a ureter of the patient.

The medial portion 230 of the device 200 includes an elongate portion or member 232. The elongate portion 232 includes a sidewall 234 that defines a lumen 236. In some embodiments, and as discussed in more detail below, the lumen 236 is in fluid communication with lumens defined by the distal end portion 210 and the proximal end portion 220 of the device 200.

In the illustrated embodiment, the surface of the sidewall 234 includes a pattern. Specifically, the sidewall 234 of the elongate portion 232 includes a first section 235 and a second section 237. The thickness of the sidewall 234 at the first section 235 (shown in FIG. 2 as X) is smaller or less than the thickness of the sidewall 234 at the second section 237 (shown in FIG. 3 as Y). In some embodiments, the thickness of the sidewall 234 at the first section 235 is half the size of the thickness of the sidewall 234 at the second section 237. In other embodiments, the thickness of the sidewall 234 at the first section 235 is less than half of the thickness of the sidewall 234 at the second section 237. In some embodiments, the thicknesses of the sidewall are determined or selected based on desired flexibility of the device. The pattern on the surface of the sidewall 234 may form any sort of pattern or shape. For example, the surface of the sidewall may include a pattern of spiral, a plurality of spirals, a random curvilinear line, or a plurality of curvilinear lines.

In the illustrated embodiment, the first section 235 of the sidewall 234 includes or forms an annular groove or ring about the elongate portion or member 232. In other embodiments, the first section 235 of the sidewall 234 includes or forms grooves that form shapes other than annular grooves or rings. In some embodiments, the annular groove or rings extend perpendicular or substantially perpendicular to a longitudinal axis of the medial portion 230 of the device 200. For example, the first section 235 may include grooves that form spiral or helical rings. In other embodiments, the first section 235 may include grooves or indentations that form circles or ovals.

In the illustrated embodiment, the sidewall 234 includes several annular grooves or rings that extend about the elongate portion or member 232. Accordingly, in the illustrated embodiment, the medial portion 230 includes a third section (a second annular groove or ring) and a fourth section (another thicker section that is disposed adjacent the second annular groove or ring). In some embodiments, the thicknesses of the sections that include the annular grooves or rings are less than the thickness of the second section 237 of the elongate member 232. The device may include any number of grooves or rings or thin sections.

In some embodiments, the thin sections of the sidewall 234 allow the medical device 200 to flex or bend to accommodate specific anatomical shapes of a patient or to conform to patient movements.

In the illustrated embodiment, the distal end portion 210 includes a retention portion or member 212. The retention portion or member 212 may be disposed within the kidney of the patient and my help retain the device 200 in place within the body of the patient. In the illustrated embodiment, the retention portion or member 212 includes a curl or coiled portion 214. In other embodiments, another type of retention portion is included at the distal end portion 210. For example, the retention portion may include flared or a different shaped portion to help retain the device 200 within the body of the patient.

In the illustrated embodiment, the distal end portion 210 defines a lumen 216 that is in fluid communication with the lumen 236 of the medial portion 230. The lumens 216 and 236 are configured to convey bodily fluid from one location within the body of the patient to another location within the body of the patient. In some embodiments, the distal end portion 210 or the medial portion 230 or both include side ports or openings that are in fluid communication with the lumens 216 and 236 and are configured to help facilitate the movement of the bodily fluid. The side ports or openings may form or define any shape. Additionally, the distal end portion 210 may be cut or include an opening that is in fluid communication with the lumen 216 to facilitate fluid flow into or out of the lumen 216.

In the illustrated embodiment, the proximal end portion 220 includes a retention portion or member 222. The retention portion or member 222 may be disposed within the bladder of the patient and my help retain the device 200 in place within the body of the patient. In the illustrated embodiment, the retention portion or member 222 includes a curl or coiled portion 224. In other embodiments, another type of retention portion is included at the proximal end portion 220. For example, the retention portion may include flared or a different shaped portion to help retain the device 200 within the body of the patient.

In the illustrated embodiment, the proximal end portion 220 defines a lumen 226 that is in fluid communication with the lumen 236 of the medial portion 230. In some embodiments, the proximal end portion 220 includes side ports or openings that are in fluid communication with the lumen 226 and are configured to help facilitate the movement of the bodily fluid. The proximal end portion 220 may be cut or include an opening of any shape that is in fluid communication with the lumen 226 and is configured to allow fluid to flow into or out of the lumen 226.

Although the illustrated embodiment illustrates the retention member 212 and retention member 222 as extending in different directions from a longitudinal axis of the device, the retention members 212 and 222 may extend in any direction from the longitudinal axis including directions that are parallel to each other.

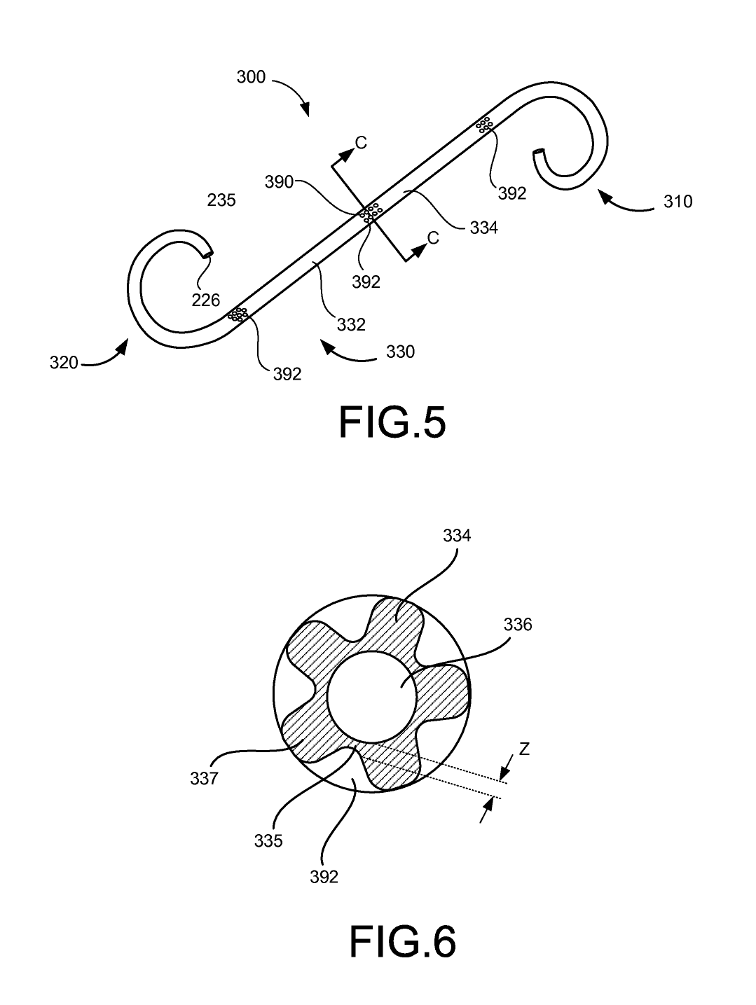

FIG. 5 is a perspective view of a medical device 300 according to an embodiment of the invention. FIG. 6 is a cross-section view of the medical device 300 taken along line C-C of FIG. 5. The medical device 300 is a ureteral stent and is configured to be disposed within a urinary tract of a patient and help transport bodily fluids from a kidney of a patient to a bladder of the patient. The medical device 300 includes a distal end portion 310, a proximal end portion 320, and a medial portion 330. In some embodiments, the medical device 300 may be placed within a body of a patient such that the distal end portion 310 may be disposed within the kidney of the patient, the proximal end portion 320 may be disposed within the bladder of the patient, and the medial portion 330 may be disposed within a ureter of the patient.

The medial portion 330 of the device 300 includes an elongate portion or member 332. The elongate portion 332 includes a sidewall 334 that defines a lumen 336. In some embodiments, the lumen 336 is in fluid communication with lumens defined by the distal end portion 310 and the proximal end portion 320 of the device 300.

The surface of the sidewall 334 includes patters of indentations, dimples, or cut-outs. The indentations, dimples, or cut-outs may include grooves, ridges, or both. The indentations, dimples, or cut-outs may have any depth, width, and length. For example, in some embodiments, the width and the length are the same or substantially the same. Specifically, in the illustrated embodiment, the sidewall 334 of the elongate portion 332 includes a portion or section 390 that includes a set or group of indentations, dimples, or cut-outs 392. In the illustrated embodiment, the sidewall 334 of the elongate portion 332 includes several portions or sections that include sets or groups of indentations or cut-outs. In the illustrated embodiment, the indentations or cut-outs 392 do not extend through the entire sidewall 334 so as to be in fluid communication with the lumen 336 defined by the sidewall 334. The indentations, dimples, or cut-outs 392 form first sections 335 of the sidewall 334 and second section 337 of the sidewall 334. The thickness of the sidewall 334 at the first sections 335 (shown in FIG. 6 as Z) is smaller or less than the thickness of the sidewall 334 at the second section 337. In some embodiments, the thickness of the sidewall 334 at the first section 335 is half the size of the thickness of the sidewall 334 at the second section 337. In other embodiments, the thickness of the sidewall 334 at the first section 335 is less than half of the thickness of the sidewall 334 at the second section 337. In some embodiments, the thicknesses of the sidewall are determined or selected based on desired flexibility of the device.

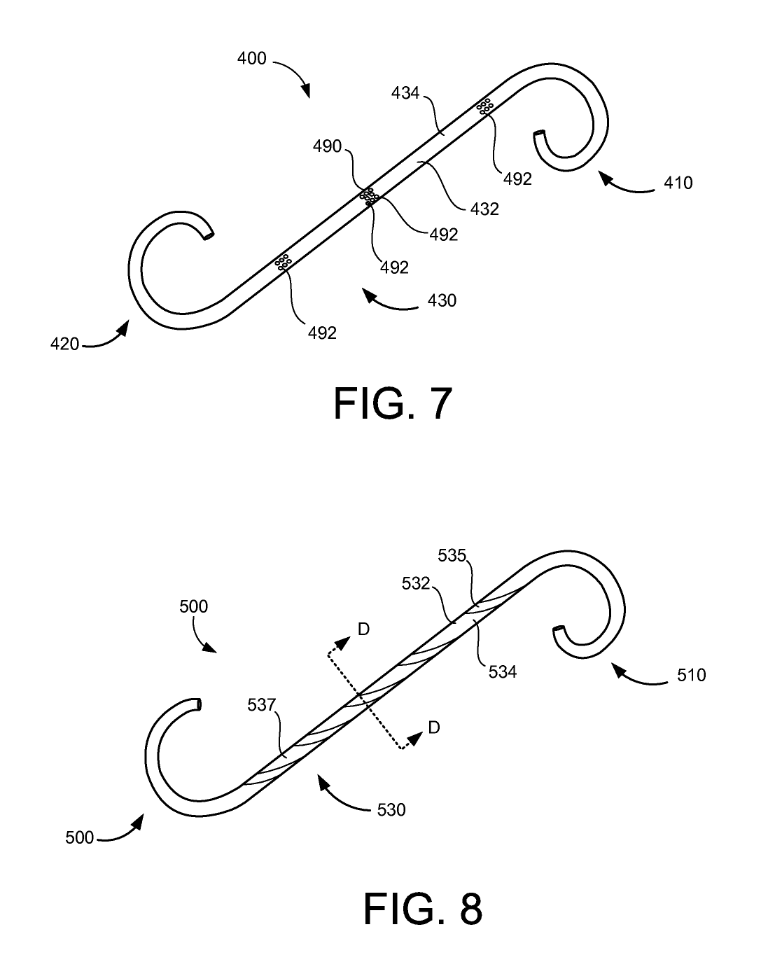

FIG. 7 is a perspective view of a medical device 400 according to an embodiment of the invention. The medical device 400 is a ureteral stent and is configured to be disposed within a urinary tract of a patient and help transport bodily fluids from a kidney of a patient to a bladder of the patient. The medical device 400 includes a distal end portion 410, a proximal end portion 420, and a medial portion 430. In some embodiments, the medical device 400 may be placed within a body of a patient such that the distal end portion 410 may be disposed within the kidney of the patient, the proximal end portion 420 may be disposed within the bladder of the patient, and the medial portion 430 may be disposed within a ureter of the patient.

The medial portion 430 of the device 400 includes an elongate portion or member 432. The elongate portion 432 includes a sidewall 434 that defines a lumen. In some embodiments, the lumen is in fluid communication with lumens defined by the distal end portion 410 and the proximal end portion 420 of the device 400.

The sidewall 434 of the elongate portion 432 includes a portion or section 490 that includes or defines a set or group 490 of openings or holes 492. The openings or holes 492 may be of any size, shape, or number. Additionally, the openings or holes may define oblong shapes and form slits or slots. In the illustrated embodiment, the sidewall 434 of the elongate portion 432 includes several portions or sections that include or define sets or groups of openings or holes. In the illustrated embodiment, the portions or sections that include or define the sets or groups of openings or holes are separated along a longitudinal axis of the device 400. In the illustrated embodiment, the openings or holes extend through the entire sidewall 434 so as to be in fluid communication with the lumen defined by the sidewall 434. In some embodiments, the openings or holes may provide increased flexibility to the stent 400. In some embodiments, the openings or holes provide for increased fluid flow into and out of the stent 400.

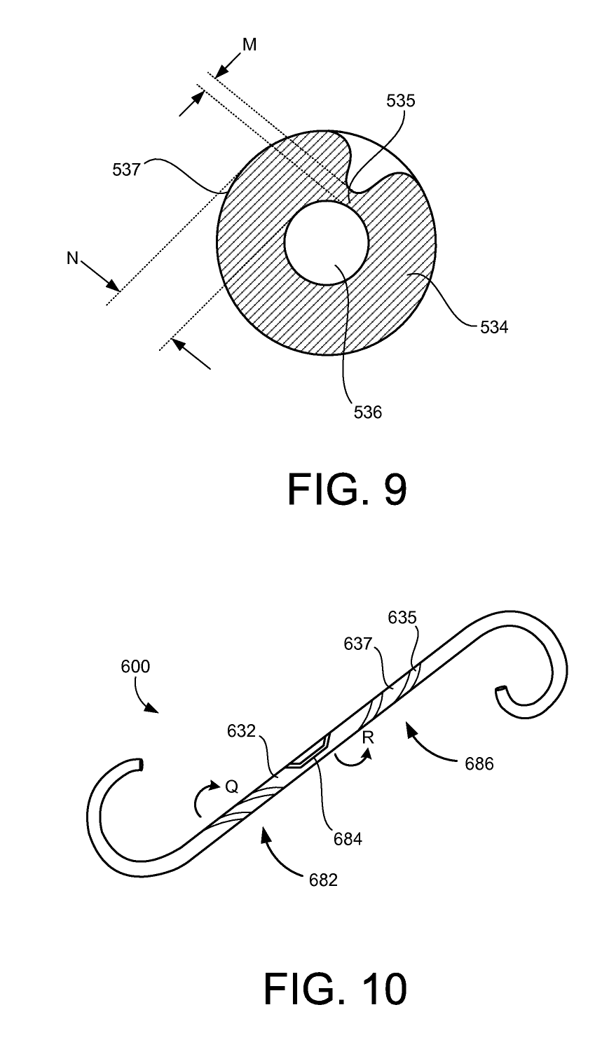

FIG. 8 is a perspective view of a medical device 500 according to an embodiment of the invention. FIG. 9 is a cross-section view of the medical device 500 taken along line D-D of FIG. 8. The medical device 500 is a ureteral stent and is configured to be disposed within a urinary tract of a patient and help transport bodily fluids from a kidney of a patient to a bladder of the patient. The medical device 500 includes a distal end portion 510, a proximal end portion 520, and a medial portion 530. In some embodiments, the medical device 500 may be placed within a body of a patient such that the distal end portion 510 may be disposed within the kidney of the patient, the proximal end portion 520 may be disposed within the bladder of the patient, and the medial portion 530 may be disposed within a ureter of the patient.

The medial portion 530 of the device 500 includes an elongate portion or member 532. The elongate portion 532 includes a sidewall 534 that defines a lumen 536. In some embodiments, the lumen 536 is in fluid communication with lumens defined by the distal end portion 510 and the proximal end portion 520 of the device 500.

In some embodiments, the surface of the sidewall 534 of the elongate portion 532 includes a pattern of grooves. The pattern may include a single groove or it may include a plurality of grooves. The pattern may include curvilinear grooves. In the illustrated embodiment, the sidewall 534 of the elongate portion 532 includes a first section 535 and a second section 537. The thickness of the sidewall 534 at the first section 535 (shown in FIG. 9 as M) is smaller or less than the thickness of the sidewall 534 at the second section 537 (shown in FIG. 9 as N). In some embodiments, the thickness of the sidewall 534 at the first section 535 is half the size of the thickness of the sidewall 534 at the second section 537. In other embodiments, the thickness of the sidewall 534 at the first section 535 is less than half of the thickness of the sidewall 534 at the second section 537. In some embodiments, the thicknesses of the sidewall are determined or selected based on desired flexibility of the device.

In the illustrated embodiment, the first section 535 of the sidewall 534 includes or forms a helical groove or spiral or ring about the elongate portion or member 532. In the illustrated embodiment, the device 500 includes only a single helical groove or ring that extends about the elongate portion or member 532. In other embodiments, the device 500 includes a plurality of helical grooves or rings that extend about the elongate portion or member or a portion of the elongate portion or member. In some embodiments, fluid may flow along the grooves or rings of the device 500. In some embodiments, fluid may flow into the lumen defined by the device via side ports or openings defined by the sidewall of the device 500.

As illustrated in FIG. 10, in one embodiment, a medical device 600 includes an elongate portion or member 632. The elongate portion or member 632 includes a first section 635 and a second section 637. The thickness of the sidewall 634 of the elongate portion or member 632 at the first section 635 is smaller or less than the thickness of the sidewall 634 at the second section 637. In some embodiments, the thickness of the sidewall 634 at the first section 635 is half the size of the thickness of the sidewall 634 at the second section 637. In other embodiments, the thickness of the sidewall 634 at the first section 635 is less than half of the thickness of the sidewall 634 at the second section 637.

In the illustrated embodiment, the first section 635 of the elongate portion or member 632 has a first section 682, a second section 684, and a third section 686. The second section 684 is disposed between the first section 682 and the third section 686. Specifically, the first section 682 is disposed proximally or towards the proximal end from the second section 684. The third section 686 is disposed distally or towards the distal end from the second section 684.

The first section 682 forms a helical groove or ring about a portion of the elongate portion or member 632. Specifically, the first section 682 forms a helical groove or ring about a portion of the elongate portion or member 632 in a first direction (illustrated as arrow Q in FIG. 10.). The helical groove or ring may be any pitch or disposed at any angle with respect to the longitudinal axis of the elongate member 632. Furthermore, in some embodiments, the pitch or angle of the helical groove or ring may change along the longitudinal length of the elongate member 632.

The third section 686 forms a helical groove or ring about a portion of the elongate portion or member 632. Specifically, the third section 686 forms a helical groove or ring about a portion of the elongate portion or member 632 in a second direction (illustrated as arrow R in FIG. 10.). The second direction is different than the first direction. In the illustrated embodiment, the second direction is opposite the first direction. For example, in one embodiment, when viewed from the proximal end portion of the device 600, the first direction is clockwise and the second direction is counter-clockwise. The first section 682 and the third section 686 may help facilitate the flexibility and the movement of the device 600 within the body of the patient as the first section 682 and the third section 686 extend different directions. The helical groove or ring may be any pitch or disposed at any angle with respect to the longitudinal axis of the elongate member 632. Furthermore, in some embodiments, the pitch or angle of the helical groove or ring may change along the longitudinal length of the elongate member 632.

The second section 684 extends between the first section 682 and the third section 684. In the illustrated embodiment, the second section 684 is linear or substantially linear. For example, the second section 684 extends in a direction parallel to a longitudinal axis of the device 600 or of the elongate portion or member 632. In other embodiments, the second section 684 extends between the first section 682 and the third section 684 and is non-linear or extends in a direction that is non-parallel to the longitudinal axis of the device 600 or of the elongate portion or member 632.

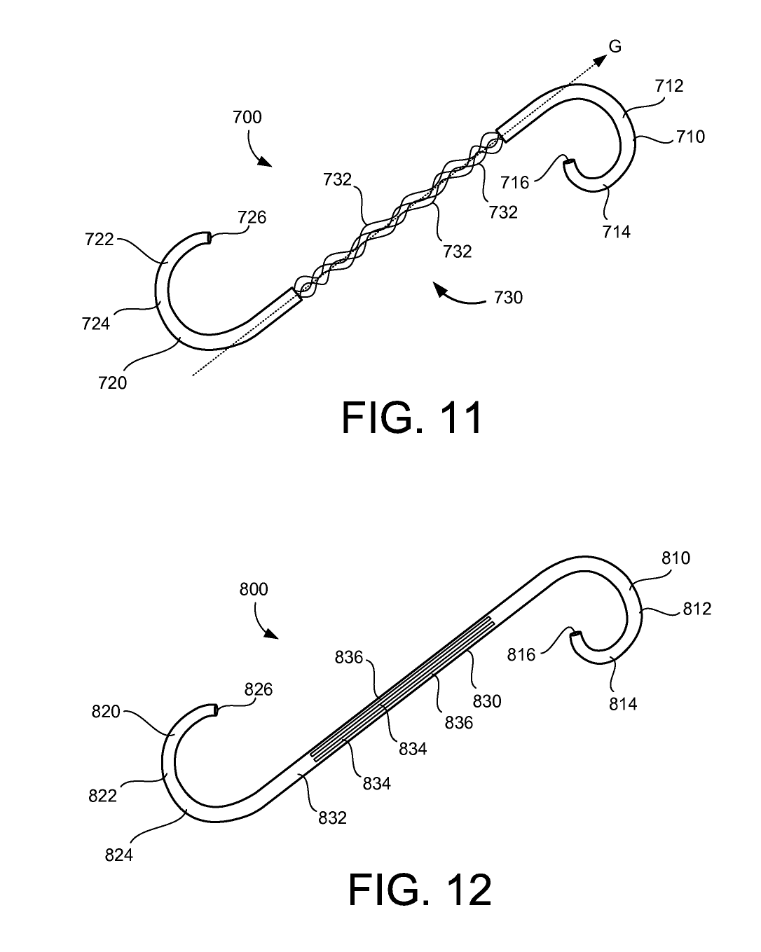

FIG. 11 is a perspective view of a medical device 700 according to an embodiment of the invention. The medical device 700 is a ureteral stent and is configured to be disposed within a urinary tract of a patient and help transport bodily fluids from a kidney of a patient to a bladder of the patient. The medical device 700 includes a distal end portion 710, a proximal end portion 720, and a medial portion 730. In some embodiments, the medical device 700 may be placed within a body of a patient such that the distal end portion 710 may be disposed within the kidney of the patient, the proximal end portion 720 may be disposed within the bladder of the patient, and the medial portion 730 may be disposed within a ureter of the patient.

In the illustrated embodiment, the distal end portion 710 includes a retention portion or member 712. The retention portion or member 712 may be disposed within the kidney of the patient and my help retain the device 700 in place within the body of the patient. In the illustrated embodiment, the retention portion or member 712 includes a curl or coiled portion 714. In other embodiments, another type of retention portion is included at the distal end portion 710. For example, the retention portion may include flared or a different shaped portion to help retain the device 700 within the body of the patient.

In the illustrated embodiment, the distal end portion 710 defines a lumen 716. The lumen 716 is configured to convey bodily fluid from one location within the body of the patient to another location within the body of the patient. In some embodiments, the distal end portion 710 includes side ports or openings that are in fluid communication with the lumen 716 and are configured to help facilitate the movement of the bodily fluid.

In the illustrated embodiment, medial portion 730 includes a plurality of strands or fibers 732 that extend from the distal end portion 710 to the proximal end portion 720. In some embodiments, the strands or fibers 732 do not include or define any internal lumen. In other words, they are each devoid of an internal lumen. In the illustrated embodiment, the strands or fibers are intertwined or braided to form a rope type structure. In some embodiments, spaces between the strands or fibers 732 (or interstices) allow fluid, such as bodily fluid, to pass through or along the medial portion 730. Accordingly, bodily fluid may pass from the distal end portion 710, along the surfaces of the strands or fibers 732 or through spaces between the individual strands or fibers 732, and to the proximal end portion 720.

In some embodiments, the strands or fibers 732 are cylindrical members and have a round or circular cross-sectional shape. In other embodiments, the strands or fibers 732 have a different shaped cross-section, such as flat or rectangular.

In some embodiments, the strands or fibers 732 are formed of a thin material. For example, in some embodiments, the strands or fibers 732 have a diameter or width of about are about 0.0005 inches (0.0013 cm) to about 0.005 inches (0.013 cm). In other embodiments, the stands or fibers 732 have larger or smaller diameters.

In the illustrated embodiment, each of the strands or fibers is coupled at one end to the distal end portion 710 and at the other end to the proximal end portion 720. Accordingly, there are no loose strands or ends of strands.

In some embodiments, the medial portion 730, including the strands or fibers 732, is flexible. In some embodiments, the medial portion 730, including the strands or fibers 732, is configured to elongate axially (i.e., along a longitudinal axis G of the medial portion 730). In some embodiments, the flexibility and the capability to elongate allows the device 700 to conform to a body of the patient.

In some embodiments, the medial portion 730, including the strands or fibers 732, does not include any stray ends or frays. In some embodiments, the medial portion 720 includes a fluoropolymer surface.

In some embodiments, the medial portion 730, including the strands or fibers 732 are formed of a wicking material. For example, in some embodiments, the strands or fibers 732 are formed of a material that is configured to help facilitate the movement of the bodily fluid. In some embodiments, the medial portion 730 is formed of a hydrophobic material.

In the illustrated embodiment, the proximal end portion 720 includes a retention portion or member 722. The retention portion or member 722 may be disposed within the bladder of the patient and my help retain the device 700 in place within the body of the patient. In the illustrated embodiment, the retention portion or member 722 includes a curl or coiled portion 724. In other embodiments, another type of retention portion is included at the proximal end portion 720. For example, the retention portion may include flared or a different shaped portion to help retain the device 700 within the body of the patient. In some embodiments, the proximal end portion 720 defines a lumen 726 that is configured to help convey bodily fluid from one location within the body to another location within the body.

Although the illustrated embodiment includes a proximal end portion 720 that includes a retention portion 722, in some embodiments, the proximal end portion 720 is devoid of a retention portion. Additionally, in some embodiments, the device 700 is devoid of a proximal end portion.

FIG. 12 is a perspective view of a medical device 800 according to an embodiment of the invention. The medical device 800 is a ureteral stent and is configured to be disposed within a urinary tract of a patient and help transport bodily fluids from a kidney of a patient to a bladder of the patient. The medical device 800 includes a distal end portion 810, a proximal end portion 820, and a medial portion 830. In some embodiments, the medical device 800 may be placed within a body of a patient such that the distal end portion 810 may be disposed within the kidney of the patient, the proximal end portion 820 may be disposed within the bladder of the patient, and the medial portion 830 may be disposed within a ureter of the patient.

In the illustrated embodiment, the distal end portion 810 includes a retention portion or member 812. The retention portion or member 812 may be disposed within the kidney of the patient and my help retain the device 800 in place within the body of the patient. In the illustrated embodiment, the retention portion or member 812 includes a curl or coiled portion 814. In other embodiments, another type of retention portion is included at the distal end portion 810. For example, the retention portion may include flared or a different shaped portion to help retain the device 800 within the body of the patient.

In the illustrated embodiment, the distal end portion 810 defines a lumen 816. The lumen 816 is configured to convey bodily fluid from one location within the body of the patient to another location within the body of the patient. In some embodiments, the distal end portion 810 includes side ports or openings that are in fluid communication with the lumen 816 and are configured to help facilitate the movement of the bodily fluid.

In the illustrated embodiment, medial portion 830 includes a tubular portion 832 that includes a ribbed portion. Specifically, the tubular portion 832 includes or defines a plurality of slits, slots, or windows 834 and forms ribs 836. In some embodiments, each of the ribs is devoid of an internal lumen. In some embodiments, the slits, slots, or windows 834 allow the device 800 to be flexible and conform to a body of a patient.

In some embodiments, the device 800, including the medial portion 830, may be formed through an extrusion process. The tubular member that is formed through the extrusion process may then be cut to form the slots or windows 834. For example, in some embodiments, a knife or other cutting device may be used to cut the extruded tubular member to form the slots or windows. In some embodiments, the slots or windows 834 are laser cut or formed by melting the material with a laser. In the illustrated embodiment, the slots or windows do not extend to the end of the device 800 (or into the proximal end portion 820).

In the illustrated embodiment, the proximal end portion 820 includes a retention portion or member 822. The retention portion or member 822 may be disposed within the bladder of the patient and my help retain the device 800 in place within the body of the patient. In the illustrated embodiment, the retention portion or member 822 includes a curl or coiled portion 824. In other embodiments, another type of retention portion is included at the proximal end portion 820. For example, the retention portion may include flared or a different shaped portion to help retain the device 800 within the body of the patient. In some embodiments, the proximal end portion 820 defines a lumen 826 that is configured to help convey bodily fluid from one location within the body to another location within the body.

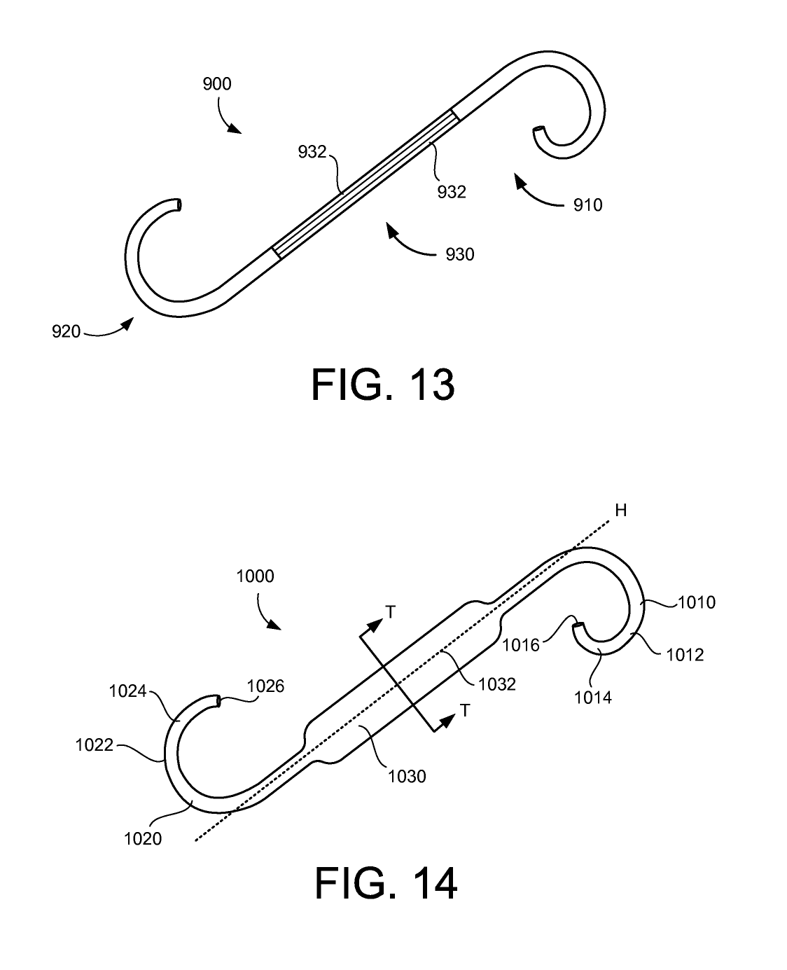

As illustrated in FIG. 13, in some embodiments, the medial portion 930 includes a plurality of strands 932 (such as bead strands) that extend between the proximal end portion 920 and the distal end portion 910. In some embodiments, each of the plurality of strands are coupled, such as via a weld, an adhesive, or other coupling mechanism, to the proximal end portion 920 and to the distal end portion 910.

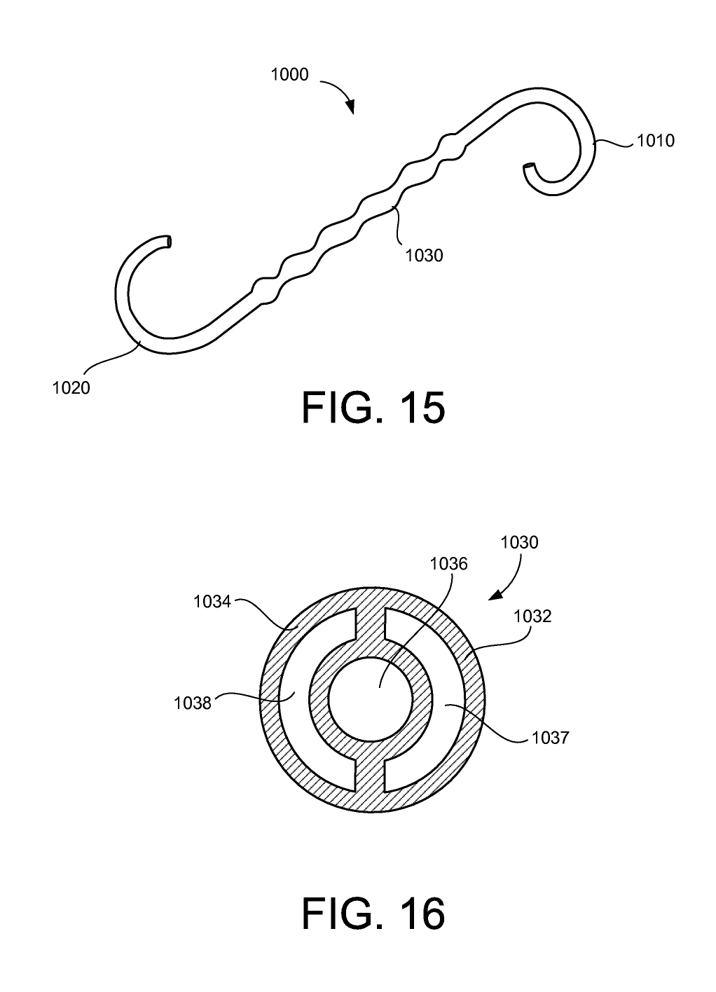

FIG. 14 is a perspective view of a medical device 1000 in a first, expanded or inflated configuration according to an embodiment of the invention. FIG. 15 is a perspective view of the medical device 1000 is a second, nominal or deflated configuration. FIG. 16 is a cross-sectional view of the medical device 1000 taken along line T-T of FIG. 14. The medical device 1000 is a ureteral stent and is configured to be disposed within a urinary tract of a patient and help transport bodily fluids from a kidney of a patient to a bladder of the patient. The medical device 1000 includes a distal end portion 1010, a proximal end portion 1020, and a medial portion 1030. In some embodiments, the medical device 1000 may be placed within a body of a patient such that the distal end portion 1010 may be disposed within the kidney of the patient, the proximal end portion 1020 may be disposed within the bladder of the patient, and the medial portion 1030 may be disposed within a ureter of the patient.

The medial portion 1030 of the device 1000 includes an elongate portion or member 1032. The elongate portion 1032 includes a sidewall 1034 that defines a lumen 1036. In some embodiments, and as discussed in more detail below, the lumen 1036 is in fluid communication with lumens defined by the distal end portion 1010 and the proximal end portion 1020 of the device 1000. The lumen 1036 is configured to help convey bodily fluid from one location within a body of a patient to another location within a body of a patient.

The sidewall 1034 of the elongate portion 1032 defines chambers 1037 and 1038. The chambers 1037 and 1038 are configured to receive a fluid or gel material to place the device 1000 in its inflated or expanded configuration (as illustrated in FIG. 14). In some embodiments, the sidewall 1034 of the elongate portion 1032 is elastic and is configured to expand or stretch when the device 1000 is in the inflated or expanded configuration. In the illustrated embodiment, the diameter or size of the medial portion 1030 is larger than the diameter or size of the proximal end portion 1020 and the distal end portion 1010. In the illustrated embodiment, the chambers 1037 and 1038 extend in a direction parallel to a longitudinal axis H of the device 1000. In some embodiments, the chambers 1037 and 1038 extend along the entire length of the medial portion 1030. In other embodiments, the chambers 1037 and 1038 extend along only a portion of the medial portion 1030. In yet further embodiments, the chambers 1037 and 1038 extend into or along a portion of either or both of the proximal end portion 1020 and the distal end portion 1010. In some embodiments, the device 1000 is placed into its expanded configuration by elastically expanding. In other words, in some embodiments, the device 1000 my expand without the use of an expansion fluid disposed within a chamber.

In some embodiments, the device 1000 is configured to be placed within the body of the patient while the device 1000 is in its inflated or expanded configuration. The fluid or gel disposed within the chambers 1037 and 1038 provide support (rigidity) to the device 1000. In some embodiments, the rigidity of the device 1000 helps facilitate the placement of the device 1000 within the body of the patient. For example, in some embodiments, the rigidity of the device 1000 helps facilitate the movement of the device 1000 along a guidewire to a location within the body of the patient.

The fluid or gel may be configured to be expelled from the chambers 1037 and 1038 to place the device 100 in its collapsed configuration (as illustrated in FIG. 15). For example, in some embodiments, once the device 1000 is placed within the body of the patient, the fluid or gel may be expelled from the chambers 1037 and 1038. In some embodiments, the device is less rigid and is more flexible when the device 1000 is in its collapsed, nominal, or deflated configuration. Accordingly, in such embodiments, the device 1000 may be placed within the body of the patient in a rigid state and convert to a flexible state once within the body of the patient.

In some embodiments, the sidewall of the medial portion 1030 (that defines the chambers 1037 and 1038) is formed of a material that allows for the fluid or gel to diffuse or leak (or dissolve or hydrolyze) out of the chambers 1037 and 1038 over a period of time. For example, the material of the sidewall may allow the fluid or gel to defuse out of the chambers 1037 and 1038 over a period of days (such as 1 to 3 days) after being inserted into the body of the patient. For example, in some embodiments, the sidewall of the medial portion 1030 is formed of a semipermeable membrane material such as cellulose acetate.

In some embodiments, the sidewall includes or defines openings that are in fluid communication with the chambers 1037 and 1038. Valves included within the chambers 1037 and 1038 or within the openings are configured to selectively or controllably release the fluid or gel from the chambers 1037 and 1038. In such embodiments, the sidewall may be formed of conventional stent materials.

In some embodiments, the chambers 1037 and 1038 may be filled with a fluid or gel such as any biocompatible gel, a thermosensitive gel, a saline solution, a drug solution, an uncrosslinked alginate or any other type of fluid or gel.

The illustrated embodiment includes two fluid chambers 1037 and 1038. In other embodiments the device includes more or less than two fluid chambers. For example, in one embodiment, the sidewall of the medial portion defines a single fluid chamber that extends coaxial with the central lumen of the device. In other embodiments, the device includes four or more fluid chambers.

In the illustrated embodiment, the distal end portion 1010 includes a retention portion or member 1012. The retention portion or member 1012 may be disposed within the kidney of the patient and my help retain the device 1000 in place within the body of the patient. In the illustrated embodiment, the retention portion or member 1012 includes a curl or coiled portion 1014. In other embodiments, another type of retention portion is included at the distal end portion 1010. For example, the retention portion may include flared or a different shaped portion to help retain the device 1000 within the body of the patient.

In the illustrated embodiment, the distal end portion 1010 defines a lumen 1016 that is in fluid communication with the lumen 1036 of the medial portion 1030. The lumens 1016 and 1036 are configured to convey bodily fluid from one location within the body of the patient to another location within the body of the patient. In some embodiments, the distal end portion 1010 or the medial portion 1030 or both include side ports or openings that are in fluid communication with the lumens 1016 and 1036 and are configured to help facilitate the movement of the bodily fluid.

In the illustrated embodiment, the proximal end portion 1020 includes a retention portion or member 1022. The retention portion or member 1022 may be disposed within the bladder of the patient and my help retain the device 1000 in place within the body of the patient. In the illustrated embodiment, the retention portion or member 1022 includes a curl or coiled portion 1024. In other embodiments, another type of retention portion is included at the proximal end portion 1020. For example, the retention portion may include flared or a different shaped portion to help retain the device 1000 within the body of the patient.

In the illustrated embodiment, the proximal end portion 1020 defines a lumen 1026 that is in fluid communication with the lumen 1036 of the medial portion 1030. In some embodiments, the proximal end portion 1020 includes side ports or openings that are in fluid communication with the lumen 1026 and are configured to help facilitate the movement of the bodily fluid.

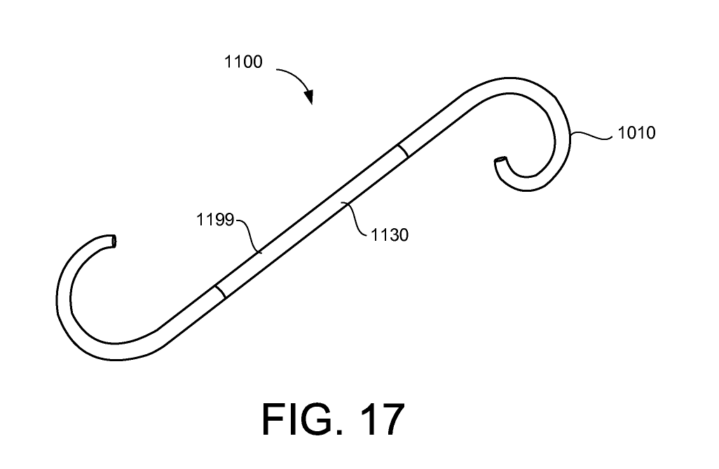

FIG. 17 is a perspective view of a medical device 1100 according to an embodiment of the invention. The medical device 1100 is a ureteral stent and is configured to be disposed within a urinary tract of a patient and help transport bodily fluids from a kidney of a patient to a bladder of the patient. The medical device 1100 includes a distal end portion 1110, a proximal end portion 1120, and a medial portion 1130. In some embodiments, the medical device 1100 may be placed within a body of a patient such that the distal end portion 1110 may be disposed within the kidney of the patient, the proximal end portion 1120 may be disposed within the bladder of the patient, and the medial portion 1030 may be disposed within a ureter of the patient.

The device 1100 includes a coating or covering 1199 disposed along a surface, such as the outer surface of the device, that is configured to provide support or rigidity to the device 1100. The coating or covering 1199 is configured to dissolve after the device 1100 is placed within the body of the patient. In such embodiments, the device 1100 may be placed within the body of the patient in a stiff or rigid state and may be configured to become flexible or soft once the device is placed within the body of the patient (after the covering or coating 1199 has dissolved).

In some embodiments, the covering or coating 1199 covers the entire outer surface for the device 1100. In other embodiments, the covering or coating 1199 covers only a portion of the outer surface of the device 1100. In some embodiments, the covering or coating 1199 is disposed on an inner surface of the device 1100 (such as on the surface that defines the internal lumen).

In some embodiments, the covering or coating 1199 is configured to dissolve after being disposed within the body of the patient for a few hours. For example, in some embodiments, the covering or coating 1199 is configured to dissolve in urine. In other embodiments, the covering or coating 1199 is configured to dissolve in blood, bile, or other bodily fluids. In other embodiments, the covering or coating 1199 is configured to dissolve after being disposed within the body of the patient for a few days. In some embodiments, the covering or coating 1199 is configured to provide additional rigidity to the device during insertion of the device into a body of a patient. In other embodiments, the covering or coating 1199 is configured to allow the device to be more flexible after placement within the body of the patient. In some embodiments, the covering or coating 1199 is disposed within the grooves or rings of the medical devices described in detail above.

In some embodiments, a coil or braid may be disposed within the sidewall of the device. The coil or braid may provide additional torsion strength and flexibility. The coil pitch may vary along the length of the device or sidewall to provide for a varied flexibility along the length of the device.

In some embodiments, a stent includes a retention portion configured to help retain the stent in place within a body of a patient; and an elongate portion extending from the retention portion, the elongate portion having a sidewall defining a lumen, the sidewall having a first section and second section, the first section of the sidewall having a first thickness, the second section of the sidewall having a second thickness different than the first thickness.

In some embodiments, the retention portion is configured to be disposed within a kidney of the patient. In some embodiments, the retention portion is a first retention potion, the stent further includes a second retention portion configured to help retain the stent in place within the body of the patient.

In some embodiments, the first section of the sidewall forms an annular ring. In some embodiments, the first section of the sidewall forms a spiral. In some embodiments, the first section of the sidewall forms a dimple.

In some embodiments, the sidewall has a third portion, the second portion of the sidewall being disposed between the first portion of the sidewall and the third portion of the sidewall.

In some embodiments, the sidewall has a third portion, the third portion having a thickness different than the second thickness, the second portion of the sidewall being disposed between the first portion of the sidewall and the third portion of the sidewall.

In some embodiments, the sidewall has a third portion, the second portion of the sidewall being disposed between the first portion of the sidewall and the third portion of the sidewall, the third portion having a third thickness, the second thickness being greater than the first thickness, the second thickness being greater than the third thickness. In some embodiments, the first portion of the sidewall has a first section and a second section, the first section of the first portion forming a spiral rotating in a first direction, the second section of the first portion forming a spiral rotating in a second direction different than the first direction.

In some embodiments, a stent includes a retention portion configured to help retain the stent in place within a body of a patient; and an elongate portion extending from the retention portion, the elongate portion having a first member and a second member, the first member being devoid of a lumen, the second member being devoid of a lumen, the first member and the second member being intertwined.

In some embodiments, the retention portion is a first retention portion, the stent further including a second retention portion, the elongate portion extending between the first retention portion and the second retention portion.

In some embodiments, the elongate portion has a third member, the first member of the elongate portion, the second member of the elongate portion, and the third member of the elongate portion being intertwined. In some embodiments, the first member of the elongate portion is connected to the retention portion. In some embodiments, the retention portion is configured to be placed within a kidney of the patient.

In some embodiments, a stent includes a retention portion configured to help retain the stent in place within a body of a patient; and an elongate portion extending from the retention portion and having an expanded configuration and a nominal configuration, the elongate portion having a sidewall defining a lumen extending from a first end portion of the elongate portion to a second end portion of the elongate portion, the sidewall defining a chamber, the chamber being configured to receive a fluid to place the elongate portion in its expanded configuration.

In some embodiments, the chamber is disposed concentrically to the lumen defined by the sidewall. In some embodiments, the chamber is a first chamber, the sidewall of the elongate portion defining a second chamber configured to receive a fluid. In some embodiments, the elongate portion has a first stiffness when the elongate portion is in its expanded configuration, the elongate portion having a second stiffness when the elongate portion is in its nominal configuration, the first stiffness being stiffer than then second stiffness.

In some embodiments, the retention portion is a first retention portion, the stent further including a second retention potion configured to help retain the stent in place within the body of the patient, the elongate portion extending from the first retention portion to the second retention portion.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A stent, comprising:
a retention portion configured to help retain the stent in place within a body of a patient; and
an elongate portion extending from the retention portion, the elongate portion having a sidewall defining a lumen, the sidewall having a first section and second section, the first section of the sidewall having a first thickness, the second section of the sidewall having a second thickness different than the first thickness.

2. The stent of claim 1, the retention portion being configured to be disposed within a kidney of the patient.

3. The stent of claim 1, the retention portion being a first retention potion, the stent further comprising:
a second retention portion configured to help retain the stent in place within the body of the patient.

4. The stent of claim 1, wherein the first section of the sidewall forms an annular ring.

5. The stent of claim 1, wherein the first section of the sidewall forms a spiral.

6. The stent of claim 1, wherein the first section of the sidewall forms a dimple.

7. The stent of claim 1, sidewall having a third portion, the second portion of the sidewall being disposed between the first portion of the sidewall and the third portion of the sidewall.

8. The stent of claim 1, the sidewall having a third portion, the third portion having a thickness different than the second thickness, the second portion of the sidewall being disposed between the first portion of the sidewall and the third portion of the sidewall.

9. The stent of claim 1, sidewall having a third portion, the second portion of the sidewall being disposed between the first portion of the sidewall and the third portion of the sidewall, the third portion having a third thickness, the second thickness being greater than the first thickness, the second thickness being greater than the third thickness.

10. The stent of claim 1, the first portion of the sidewall having a first section and a second section, the first section of the first portion forming a spiral rotating in a first direction, the second section of the first portion forming a spiral rotating in a second direction different than the first direction.

11. A stent, comprising:
a retention portion configured to help retain the stent in place within a body of a patient; and
an elongate portion extending from the retention portion, the elongate portion having a first member and a second member, the first member being devoid of a lumen, the second member being devoid of a lumen, the first member and the second member being intertwined.

12. The stent of claim 11, the retention portion being a first retention portion, the stent further comprising:
a second retention portion,
the elongate portion extending between the first retention portion and the second retention portion.

13. The stent of claim 11, the elongate portion having a third member, the first member of the elongate portion, the second member of the elongate portion, and the third member of the elongate portion being intertwined.

14. The stent of claim 11, wherein the first member of the elongate portion is connected to the retention portion.

15. The stent of claim 11, the retention portion being configured to be placed within a kidney of the patient.

16. A stent, comprising:
a retention portion configured to help retain the stent in place within a body of a patient; and
an elongate portion extending from the retention portion and having an expanded configuration and a nominal configuration, the elongate portion having a sidewall defining a lumen extending from a first end portion of the elongate portion to a second end portion of the elongate portion, the sidewall defining a chamber, the chamber being configured to receive a fluid to place the elongate portion in its expanded configuration.

17. The stent of claim 16, the chamber being disposed concentrically to the lumen defined by the sidewall.

18. The stent of claim 16, the chamber being a first chamber, the sidewall of the elongate portion defining a second chamber configured to receive a fluid.

19. The stent of claim 16, the elongate portion having a first stiffness when the elongate portion is in its expanded configuration, the elongate portion having a second stiffness when the elongate portion is in its nominal configuration, the first stiffness being stiffer than then second stiffness.

20. The stent of claim 16, the retention portion being a first retention portion, the stent further comprising:
a second retention potion configured to help retain the stent in place within the body of the patient,
the elongate portion extending from the first retention portion to the second retention portion.
